(19) United States
(12) Patent Application Publication
(10) Pub. No.: US 2022/0062705 A1
(43) Pub. Date: Mar. 3, 2022

(54) ULTRASONIC WAVE SYNCHRONIZATION

(71) Applicant: MotivateU, Inc., Middletown, DE (US)

(72) Inventor: Jennifer K. Strout, Virginia Beach, VA (US)

(21) Appl. No.: 17/463,312

(22) Filed: Aug. 31, 2021

Related U.S. Application Data
(60) Provisional application No. 63/072,857, filed on Aug. 31, 2020.
(60) Provisional application No. 63/074,978, filed on Sep. 4, 2020.
(60) Provisional application No. 63/134,029, filed on Jan. 5, 2021.

(51) Int. Cl. A63B 24/00; G01S 15/06

(52) U.S. Cl. CPC A63B 24/0062; A63B 24/0021; A63B 2024/0025; A63B 2024/0068; G01S 15/06

(57) ABSTRACT

An ultrasonic wave synchronization system may employ in-air sonar to monitor the location and movements of targets during a session. The system utilizes the signals' properties to determine whether the targets are in synchronization with one another's positioning. An instructor may track whether a participant or trainee is in synchronization with the instructor's movements, thus determining whether the participant is performing the movements properly and in synchronization with the instructor. Further instruction may be given to the participant to allow the participant to come into synchronization with the instructor. The acoustic signal may be filtered to remove any unwanted acoustic signals created by the environment. A user interface may be provided to allow the users to calibrate, indicate settings, monitor the session, monitor progress during and/or after the session, and provide other information to the users.

### CROSS-REFERENCE TO RELATED APPLICATIONS

This application claims priority to U.S. Provisional Application No. 63/072,857 filed Aug. 31, 2020, U.S. Provisional Application No. 63/074,978 filed Sep. 4, 2020, and U.S. Provisional Application No. 63/134,029 filed Jan. 5, 2021, the contents of each of which are incorporated by reference in their entireties.

### TECHNICAL FIELD

This patent application relates generally to ultrasonic wave synchronization. In particular, the application relates to ultrasonic wave synchronization between users through computing devices.

### BACKGROUND

Currently, fitness classes, programs, and training are provided in person at studios, fitness centers, and gyms, etc. The participants gather at a studio, for example, with one or more trainers, instructors, or fitness guides. The trainers lead the participants in a group exercise. Such group exercise or training provides motivation to participants, assists participants in being held accountable, and thus sticking to their goals, provides a sense of community and friendship, and assists participants in staying on course with their fitness routine. Some people may be unable to access the gyms due to physical limitations or other reasons (e.g., pandemics). Without this access to gyms, some people may become isolated, lonely, and may lack physical fitness, all of which may lead to poor health.

To address the difficulties with in-person classes, several applications (“app”) or web-based solutions exist. For example, Peloton®, Mirror®, and Tempo® all provide users the ability to exercise in the convenience of their own homes while still having access to group fitness instruction. However, these solutions require specific and expensive hardware and/or equipment as well as recurring memberships. The equipment is not mobile, but rather is bulky and unable to be easily transported (e.g., large exercise bicycles, treadmills, and full-length mirrors). These solutions are not truly community based (virtual community only). Individuals cannot link their app profiles and classes with their current gym/fitness center. These systems lack live feedback from human fitness instructor and in-group psychology with the related motivation to commit to a positive habit. All current electronic solutions are individual based, which drives up isolation and loneliness.

Another approach to addressing the limitations of in-person instruction involve community fitness center solutions. This involves live web streamed classes, similar to watching a fitness video on the television. Many thousands of free fitness videos exist on web platforms. The reliability and availability is subject to strong internet connection and there is no individualized live feedback from human fitness instructor as an instructor cannot hold the class while simultaneously watching a multitude of browser windows to determine who is accurately and safely participating in-sync with the session.

Therefore, a need exists for a community centered, app-based fitness program. Specifically, a need exists for a fitness system that includes synchronization between users, for example between a trainer and participant, through mobile devices.

### BRIEF SUMMARY

According to an embodiment of the disclosure, an ultrasonic wave synchronization system may include an acoustic system configured to measure a first parameter of a first user and a second parameter of a second user, wherein the first parameter is compared to the second parameter to determine a percentage of synchronization between the first user and the second user.

According to an embodiment of the disclosure, the acoustic system comprises a first transducer configured to emit and receive ultrasonic sound waves and a second transducer configured to emit and receive ultrasonic sound waves.

According to an embodiment of the disclosure, the first parameter and the second parameter may result from ultrasonic sound waves reflecting off the first user and the second user, respectively, or may result from another form of motion capture of the users' parameters.

According to an embodiment of the disclosure, the first parameter is compared to the second parameter to determine closeness of the parameters and wherein the percentage of synchronization is representative of the closeness of the parameters.

According to an embodiment of the disclosure, the acoustic system may be an in-air sonar system or a doppler system.

According to an embodiment of the disclosure, the first parameter and the second parameter are one or more of angularity, frequency, velocity, and acceleration and wherein the acoustic system measures the first and second parameter by transmitting and receiving ultrasonic soundwaves or another form of motion capture of the users' parameters.

According to an embodiment of the disclosure, the first user is an instructor, and the secondary user(s) are one or more participants, and wherein the percentage of synchronization is representative of an amount of synchronization of body movements of each of the one or more participants with respect to body movements of the instructor during a fitness class.

According to an embodiment of the disclosure, the instructor is alerted to at least one of the one or more participants that are in synchronization with the instructor below a predetermined target percentage of synchronization.

According to an embodiment of the disclosure the predetermined synchronization target range may be selected to the default range or may be customized by the instructor prior to the fitness class.

According to an embodiment of the disclosure, the threshold for the percentage of synchronization that results in an alert is higher for a beginner level fitness class than an advanced level fitness class.

According to an embodiment of the disclosure, the default settings are that when the percentage of synchronization is below 50% a red alert will display, a synchronization range between 50-69% is neutral and results in an orange display, and a synchronization percentage of 70% or above is a satisfactory and results in a green display.

According to an embodiment of the disclosure, the acoustic system may further comprises a transmitter and two receivers for each of the first user and the second user.

According to an embodiment of the disclosure, the transmitter and two receivers for the first user may be located at the same respective location to the first user as the transmitter and two receivers of the second user.

According to an embodiment of the disclosure, the first parameter is compared to the second parameter in real-time.

According to an embodiment of the disclosure, the first parameter is compared to the second parameter in real-time such that the comparing occurs simultaneously and continuously while the first user and the second user are engaged in an activity.

According to an embodiment of the disclosure, an ultrasonic wave synchronization system may include a first in-air sonar system, comprising: a first transmitter configured to transmit a first ultrasonic soundwave; and a first receiver configured to receive the first ultrasonic soundwave, wherein the first in-air sonar system is configured to measure a first parameter of a first user; a second in-air sonar system, comprising: a second transmitter configured to transmit a second ultrasonic soundwave; and a second receiver configured to receive the second ultrasonic soundwave wherein the second in-air sonar system is configured to measure a second parameter of a second user, wherein the first parameter is compared to the second parameter to determine a percentage of synchronization between the first user and the second user.

According to an embodiment of the disclosure, the first parameter and the second parameter are one or more of angularity, frequency, velocity, and acceleration.

According to an embodiment of the disclosure, the first receiver may be comprised of two receivers and the second receiver comprises two receivers.

According to an embodiment of the disclosure, the first in-air sonar system may be remote from the second in-air sonar system.

According to an embodiment of the disclosure, the first user may be remote from the second user.

According to an embodiment of the disclosure, the first user is an instructor, and the secondary user(s) are one or more participants, and wherein the percentage of synchronization is representative of an amount of synchronization of body movements of each of the one or more participants with respect to body movements of the instructor during a fitness class.

According to an embodiment of the disclosure, the instructor may be alerted to at least one of the one or more participants that are in synchronization with the instructor below a predetermined target.

According to an embodiment of the disclosure the predetermined synchronization target range may be selected to the default range or may be customized by the instructor prior to the fitness class.

According to an embodiment of the disclosure, the parameters of the primary target or user is compared to the parameters of the secondary target(s) or user(s) in real-time.

According to an embodiment of the disclosure, a computer-implemented method of determining synchronization between users may include (a) transmitting and receiving a first acoustic signal; (b) determining a first parameter of a first user based on the received first acoustic signal; (c) transmitting and receiving a second acoustic signal; (d) determining a second parameter of a second user based on the received second acoustic signal; (e) comparing, with a processor, the first parameter to the second parameter to determine whether the first parameter and the second parameter are within a predetermined range of one another; and (f) determining, with the processor and based on the comparison, a percentage of synchronization of the first user and the second user.

According to an embodiment of the disclosure, the method may include alerting one or both the first user and the secondary user(s) when the percentage of synchronization is outside of a predetermined range.

According to an embodiment of the disclosure, the alerting is a visual, audio, tactile cue or some combination herein.

According to an embodiment of the disclosure, the method may include continuously performing steps (a)-(f).

According to an embodiment of the disclosure, the method may include steps (a)-(f) are performed in real-time.

According to an embodiment of the disclosure, the method may include steps (a)-(f) are repeated during a timed session and the percentage of synchronization is determined continuously through the timed session.

According to an embodiment of the disclosure, the method may include graphing the percentage of synchronization as compared to a predetermined target is presented during and/or after the timed session and providing a resulting graph to at least one of the first user and the second user.

According to an embodiment of the disclosure, the first an instructor, and the secondary user(s) are one or more participants of the fitness class, and wherein the percentage of synchronization is representative of an amount of synchronization of body movements of each of the one or more participants with respect to body movements of the instructor during the fitness class.

According to an embodiment of the disclosure, the method may include tracking an ability of the one or more participants to achieve synchronization within a predetermined range over the course of one or more fitness classes.

According to an embodiment of the disclosure, the method may include alerting the instructor (trainer) and/or participant (trainee) when at least one of the one or more participants is below a predetermined target of synchronization for the fitness class.

According to an embodiment of the disclosure, the first parameter and the second parameter are one or more of angularity, frequency, velocity, and acceleration.

According to an embodiment of the disclosure, the method may include calibrating a transmitter and receiver of the first user to remove noise present in the first acoustic signal, the noise resulting from an environment surrounding the first user; and calibrating a transmitter and receiver of the second user to remove noise present in the second acoustic signal, the noise resulting from an environment surrounding the second user.

According to an embodiment of the disclosure, the method may include a transmitter and receiver for the first user and a transmitter and receiver for the second user, wherein the transmitter and receiver of the first user may be located at the same respective location to the first user as the transmitter and receiver of the second user.

According to an embodiment of the disclosure, the first computing device is a personal computer, a mobile phone, or a tablet and wherein the second computing device is a personal computer, a mobile phone, or a tablet.

According to an embodiment of the disclosure, a method for determining synchronization between multiple users may include providing an in-air sonar system or other method of capture to monitor the multiple users, and comparing, with a processor, the users' parameters to determine a percentage of synchronization between at least two of the multiple users.

### BRIEF DESCRIPTION OF THE DRAWINGS

The foregoing and other features and advantages will be apparent from the following, more particular, description of various exemplary embodiments, as illustrated in the accompanying drawings, wherein like reference numbers generally indicate identical, functionally similar, and/or structurally similar elements.

FIGS. 1A and 1B are schematics of a user interface, according to an embodiment of the present disclosure.

FIG. 2 is a schematic of a user interface, according to an embodiment of the present disclosure.

FIGS. 3A and 3B are schematics of a user and a motion capture synchronization system, according to an embodiment of the present disclosure.

FIGS. 4A and 4B are schematics of a first and second user during utilization of a motion capture synchronization system, according to an embodiment of the present disclosure.

FIGS. 5A-5D are a flow chart showing a process of a motion capture synchronization system, according to an embodiment of the present disclosure.

FIG. 5E is a process chart showing process steps of a motion capture synchronization, according to an embodiment of the present disclosure.

FIG. 6 is a schematic of a user interface including a graph showing user statistics, according to an embodiment of the present disclosure.

FIGS. 7A and 7B are schematics of location of a computing device of a motion capture synchronization system, according to an embodiment of the present disclosure.

### DETAILED DESCRIPTION

Various embodiments of the invention are discussed in detail below. While specific embodiments are discussed, this is done for illustration purposes only. A person skilled in the relevant art will recognize that other components and configurations may be used without departing from the spirit and scope of the invention.

The motion capture synchronization system of the present disclosure may use in-air sonar or other form of capture to monitor the location and movements of users (e.g., participants, fitness members, instructors, trainers, etc.) during a session, such as a fitness session. The in-air sonar system emits acoustic signals and receives the signals with a device to measure or detect directionality, angularity, frequency, velocity, and/or acceleration. These values are then compared between users. The system uses the parameters' values to determine whether the users are in synchronization with the primary target's movements. For example, an instructor may track whether a participant or fitness member is in synchronization with the instructor's movements, thus determining whether the participant is performing the exercises properly and in sync with the instructor. Further instruction may be given to the participant to allow the participant to come into synchronization with the instructor. The acoustic signal may be filtered to remove any unwanted acoustic signals created by the environment. A user interface may be provided to allow the users to calibrate, indicate settings, monitor the session, monitor progress during and/or after the session, and provide other information to the users. The system allows for the instructor and participants to be remote from one another while still encouraging interaction and community environment.

The ultrasonic wave synchronization system of the present disclosure describes a system which compares the signals of ultrasonic waves to determine synchronization therebetween. That is, for example, the system emits ultrasonic sound waves from a transducer or transmitter. The ultrasonic sound waves are received by the transducer or a receiver. A signal representative of the received ultrasonic wave is then compared to a signal from another system (also having a transmitter and receiver). The comparison results in a determination of whether the two sets of parameters' values are synchronized. In practice, this may be used in a virtual fitness session, where the primary set of values is representative of the instructor and the secondary set of values from the another system is representative of a participant. The comparison, or synchronization, may be representative of how closely the participant mirrors the moves and speed of the instructor to determine the correctness of the participant's form in real-time.

In accordance with the principles of the disclosure, a system is provided that uses ultrasonic soundwave technology (e.g., in-air sonar, doppler) or other forms of wave propagation such as light (e.g. LADAR, LiDAR) or radio (e.g. RADAR) or any combination thereof to compare one set of time-dependent velocities from a first object with another set of time-dependent velocities from a second object with the purpose of determining if the two objects are in-synchronization. For example, the system may provide for using in-air sonar to determine the velocity and relative location of a primary object or user: the instructor or fitness trainer, and comparing the primary values to the velocity and relative location of a secondary object or user: the participant or trainee. This comparison allows both the instructor and participant to become aware of whether the participant is correctly performing exercises and is maintaining accuracy, speed, and/or synchronization with the instructor.

The in-air acoustic signals may measure or detect directionality, angularity, frequency, velocity, acceleration and/or spectral properties. The results of these variables are then compared between the participant(s) and the instructor to determine a percentage of synchronization between the participant(s) and the instructor.

The soundwaves or acoustic signals of the present disclosure are of ultrasonic frequency. Ultrasonic frequency signals are used to prevent the system from interfering or being affected by other sounds within the range of human frequency, from distracting the users or other humans nearby. This is accomplished due to the fact that the ultrasonic soundwaves are inaudible to humans. Ultrasound or higher spectral frequency waves may also improve the data of the input variables.

In an exemplary implementation of the system, one or more participants and an instructor, in separate, remote locations, are participating in an exercise session. The instructor performs moves, poses, and exercises, which the participants mimic. The ultrasonic wave synchronization of the present disclosure emits and receives acoustic signals with the instructor and participants' respective devices. The system compares these results to determine a percentage of synchronization between the instructor and each individual participant. The system may display the results to the instructor and/or participant(s) in real-time. The instructor may then monitor a participant who is falling behind or performing an exercise or routine incorrectly and provide appropriate instruction and/or modification to the exercise program as needed, thus leading to increased participant motivation and correction of potentially injurious athletic form.

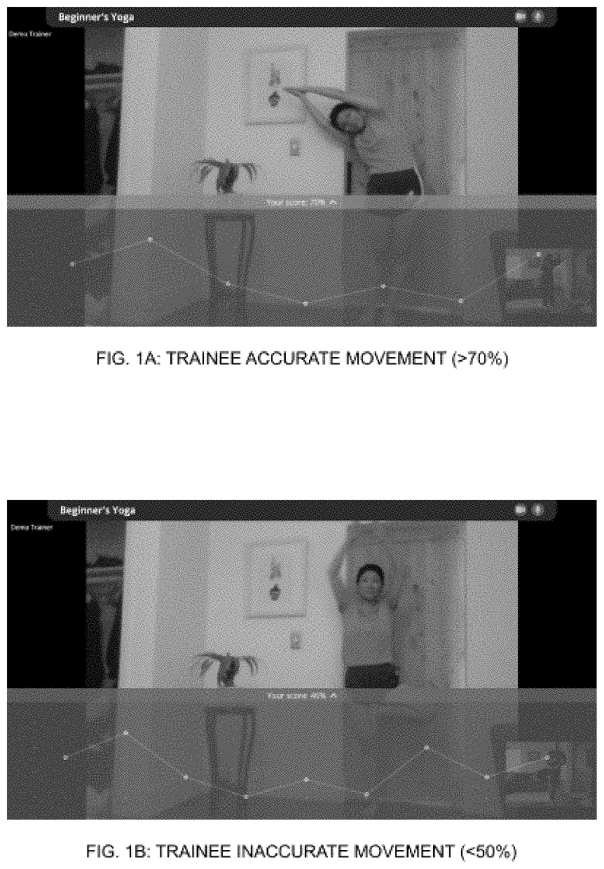

In embodiments of the disclosure, a system is provided to allow for a group/community feel and benefits without the inconvenience, risk, and/or other difficulties associated with travel to an in-person location. Participants (e.g., fitness members and instructors/trainers) may sign into their fitness classes, training sessions, or other program through existing video conference software. The system provides the instructor and each participant with real time alerts (as shown in FIGS. 1A-1B) regarding each of the participants in the class or program.

As shown in FIGS. 1A-1B, the real time alerts may indicate one or more participants logged into the program, may indicate one or more participants exceeding the preset or predetermined target goal (FIG. 1A), and/or may indicate one or more participants that are falling behind a preset or predetermined target goal (FIG. 1B). The predetermined target to which an individual participant aspires may be a percentage of synchronization between the individual participant(s) and the instructor. The percentage of synchronization may be indicative of the amount of time an individual participant mirrors the instruction with his or her body, both in speed of the movements and location of the body and/or may be indicative of the portion or percentage of the body that is aligned with the instructor's body during the session. The percentage of synchronization may be measured with an in-air sonar system or other form of data capture. The predetermined target may be preset or selected prior to the beginning of the class or program. The predetermined target may be updated or changed as needed, including during the class, by the participant and/or by the instructor. The predetermined target may be based on the class level, class size, participant fitness level, complexity or the difficulty of the class, the instructor's preference, or any combination thereof.

With continued reference to FIGS. 1A-1B, the instructor and participants may select the desired notifications to be received on the instructor's device (e.g., mobile device or computer) during and/or after a session. The participant may choose to view the alert based on the cutoff levels set by instructor, with a green bar indicating satisfactory performance, or in-synchronization with the instructor (FIG. 1A), an orange bar indicating neutral performance, and a red bar indicating unsatisfactory, or not in-synchronization with the instructor (FIG. 1B).

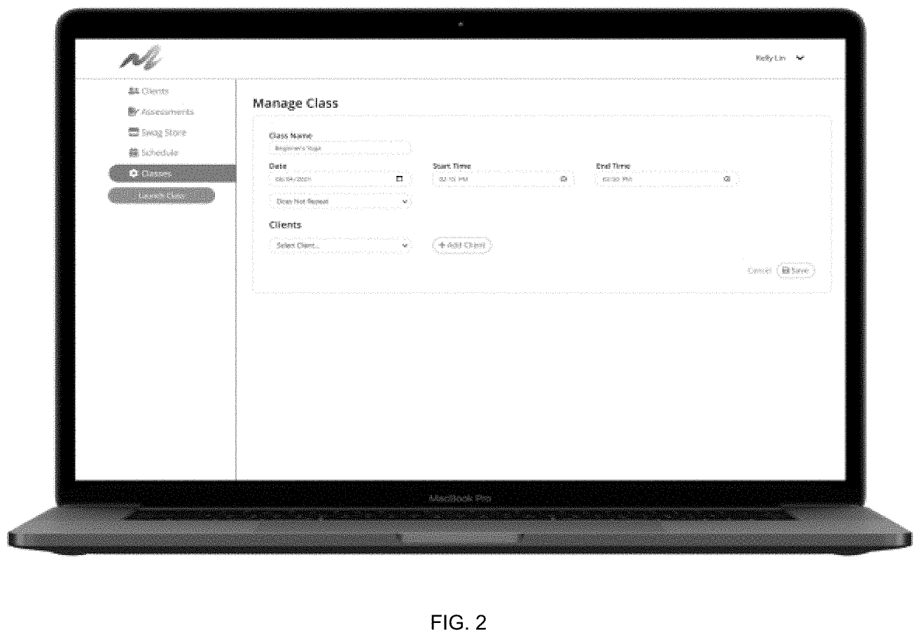

As shown in FIG. 2, the instructor may create new classes and then set the predetermined target before each class (with an option to save settings for future classes of the same name) either through a pre-formulated option based on class type (e.g., Boot Camp, HIIT, Yoga, etc.) and/or skill level (e.g., Beginner, Basic, Moderate, Advanced, Expert) or through customized settings. The customized settings may consider the difficulty of the class, the fitness level of the participant, the duration of the class, the number of the classes within a series, etc., or any combination thereof. The customized setting may allow the instructor to set a percentage of synchronization. In some embodiments, the percentage of synchronization may be estimated or calculated based on one or more of the aforementioned settings (e.g., class type, skill level, fitness level, class duration, class difficulty, etc.).

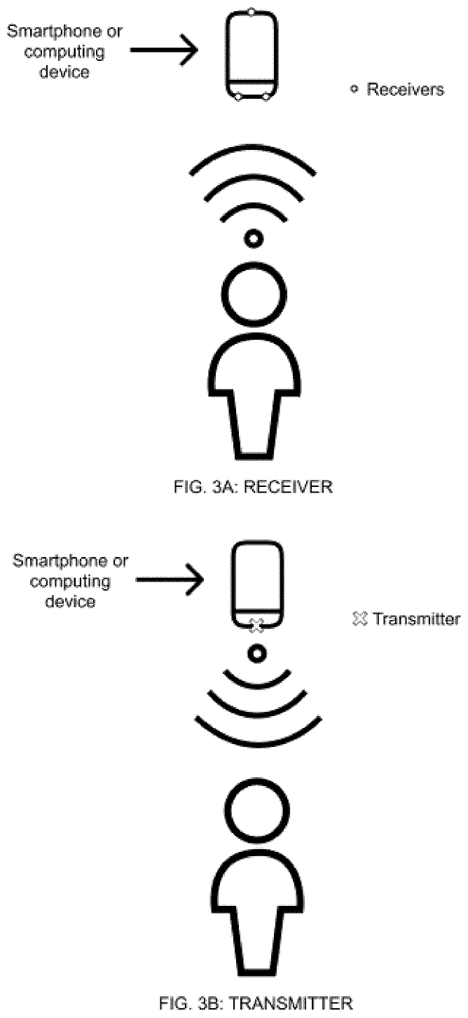

Referring to FIGS. 3A-3C, once setup is completed by both the instructor and the participant, the system will track the movements of the instructor and fitness members in real-time using in-air sound navigation and ranging (sonar), or another form of data capture. That is, the in-air sonar system may emits ultrasonic acoustic signals/waves from a transmitter (e.g., a mobile device, dedicated transmitter, and/or computer). The acoustic signals may reflect or bounce off objects within the room, including the users (e.g., the participants and the instructor), in their respective locations. The signal may then be received at a receiver(s). Although described herein with a both a transmitter and receiver, the signals may be transmitted and received by a transducer. The transducer may be capability of acting as both the transmitter and receiver. The receiver may be the same device as the transmitter and/or may be a dedicated receiver. In embodiments, two or more receivers may be present. Two or more receivers may allow for the system to increase accuracy in terms of the measured acoustic features of reflected ultrasonic soundwaves (amplitude, intensity, pulse rate, temporal, spectral features, etc.). This may occur because the intensity, frequency, temporal properties and velocity of the ultrasonic sound waves may differ at each receiver based on the target object's distance and angular positioning away from each respective receiver. The acoustic signals may be filtered to remove noise created by objects within the user's surroundings, as will be discussed in more detail to follow. The system may then compare the data received to determine whether the participant is in-sync (e.g., performing the same movements at the same time and in proper form) with the instructor. The system may determine to what degree the participant is not in-sync with the instructor (e.g., the percentage of synchronization discussed above). Indications of percentage of synchronization may be output (either visually or through audio or tactile cues) to the users.

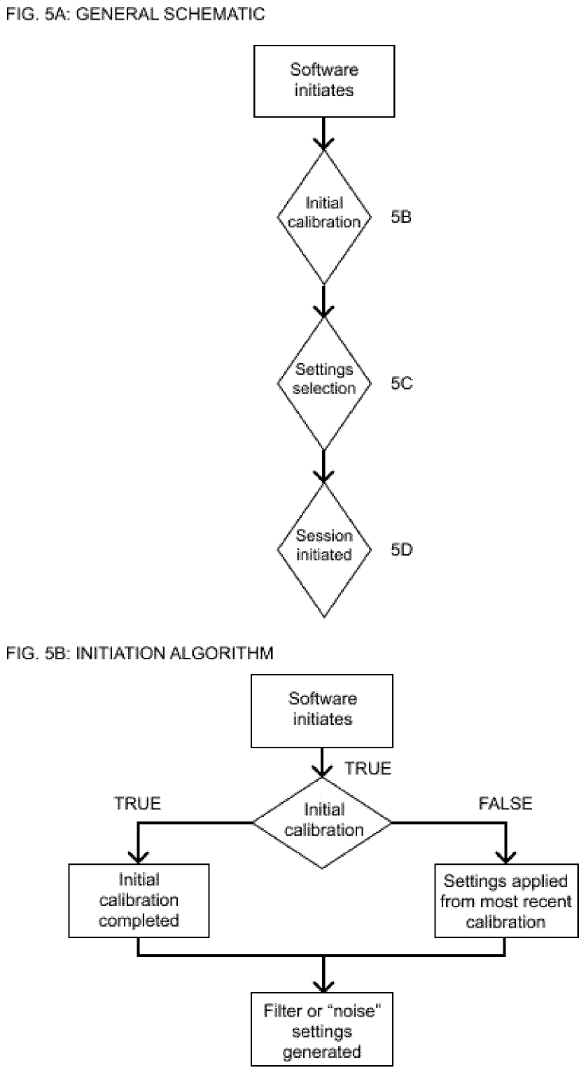

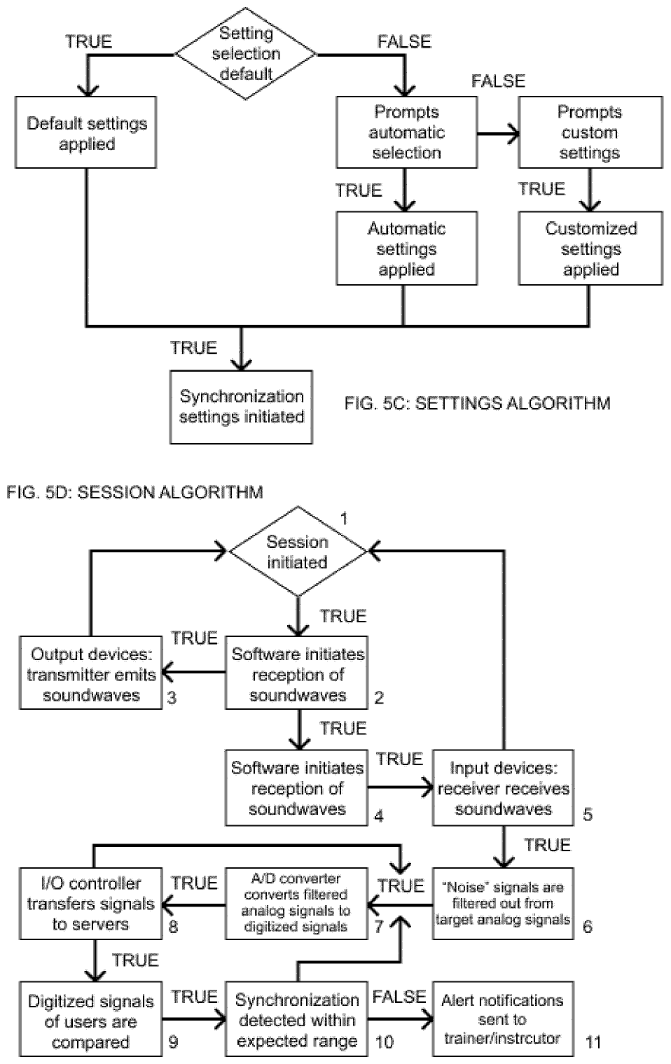

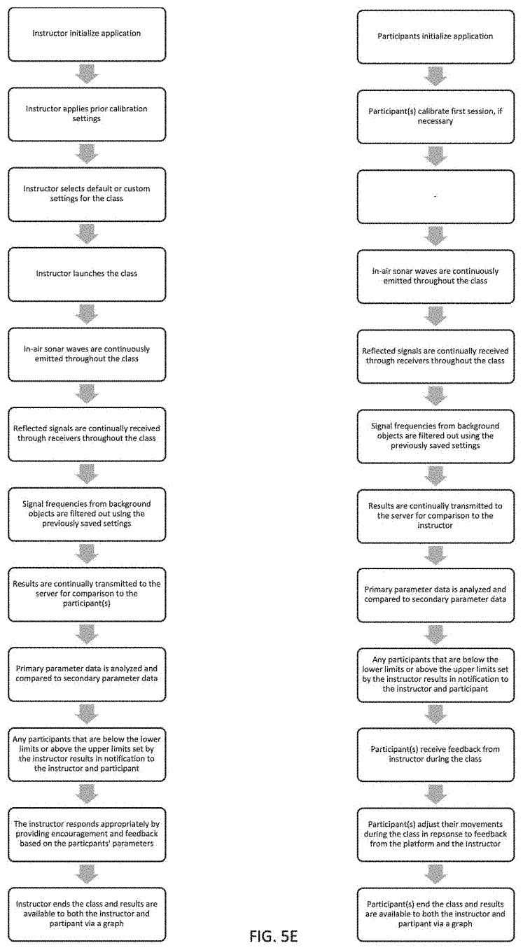

As shown in FIGS. 3A and 3B, the users' (e.g., participants and/or trainer/instructor) computing device (e.g., mobile phone, personal computer, or tablet) may include one or more transmitters and/or one or more receivers. In an embodiment, one transmitter and two receivers are present, however more may be provided. The transmitters and/or receivers may be capable of emitting and receiving, respectively, one or more acoustic signals. For example, in FIG. 3A, one or more of the receivers located within the computing device may be sonar receivers and may receive acoustic signals from the user. In the example of FIG. 3B, the computing device may include one or more sonar transmitters that may emit one or more acoustic signals. During utilization, the computing device may emit an acoustic signal, which may reflect off of the user and be received by the computing device's receiver(s). The computing device may then process the acoustic signal to determine the percentage of synchronization, such as described with respect to FIG. 5.

The system may alert the instructor, in real-time, of the percentage of synchronization of the participant(s). That is, if a participant is not meeting the pre-formulated goals and/or if the participant is meeting and/or exceeding the pre-formulated goals. For example, in a beginner's yoga, an instructor may only select to be alerted if the participants fall below a synchronization level of 20%. Thus, the system will only alert the instructor when the participants are not in-sync for this preselected amount of the time. In some embodiments, the participant may also be notified of the percentage of synchronization with the instructor.

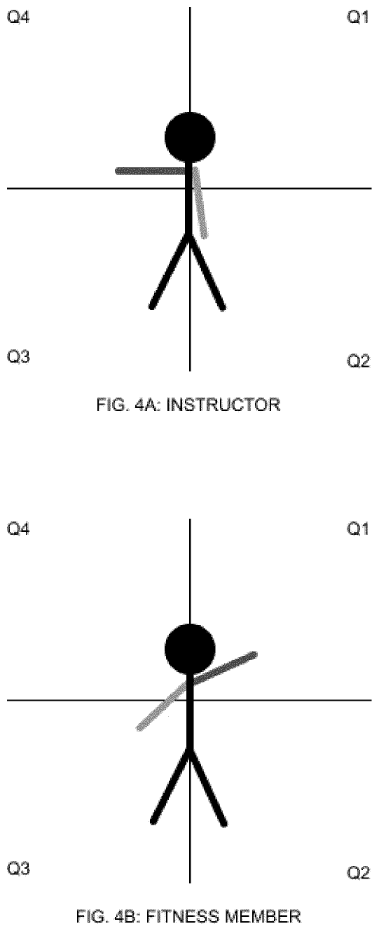

The system may be configured to ignore or not flag certain discrepancies in synchronization based on the desired percentage of synchronization desired. For example, in the example of the beginner's yoga class, the system may not flag cross-quadrant distribution of limbs. Referring to FIGS. 4A and 4B, for example, a fitness instructor's right arm may be up above his head at a 90-degree angle with his head while his left arm is down at his side at a parallel with his body (FIG. 4A), while a participant's left arm is above her head at a 45 degree angle to her head and her right arm is down at her side at a 45 degree angle to her body (FIG. 4B). The mirror reflection of the participants may not be recorded as out of synchronization while the differences in relative angular location of the limbs may be recorded as out of synchronization. Thus, referring again to FIGS. 4A and 4B, computer synchronization may accept a mirror reflection as in-synchronization: for example Quadrant 1 may be reversed with Quadrant 4 and Quadrant 2 may be interchanged with Quadrant 3, but Quadrant 1 should not be interchangeable with Quadrant 3, and Quadrant 2 should not be interchangeable with Quadrant 4.

The system may complete a setup period prior to each session. The setup period may be brief if the user (e.g., participant or instructor) is located in the same position, in the same room, without altering anything in the background of the user as in the previous session. If the user has altered anything in the background of the user's frame of reference the system may need to recalibrate during the initial setup period. In versions of the system which are absent of artificial intelligence and machine learning, it is likely, that unless the user completes workouts in front of a wall with no objects, that a setup period is required before each session. The setup period considers minor adjustments in the environment, such as, for example, objects on/in front of the wall that would require calibration would include, but are not be limited to: pictures, frames, bookshelves, dressers, refrigerators, furniture, decorations, lamps, etc. The calibration allows for the room to be configured so that going forward through the session the in-air sonar only recognizes the movements of the instructor and participants while disregarding the signals associated with the surrounding objects within the room. In some embodiments, the system may forgo the calibration by utilizing differences in the distance from the transmitter between the user and background, as well as the velocity of the user against a static background. The system may require a first/primary iteration due to small frame of reference between objects due to a smaller space being utilized during workouts (including, but not limited to hotel rooms and studio apartments). The decorations and furniture and other background objects may be “ignored” or filtered out by the program as “noise.” Such filtering of noise provides a more accurate reading of whether the participant and instructor are in-sync with one another. Alternatively, computer programming filters may be able to overcome the requirement of initial calibration and/or may be able to dramatically reduce its duration.

The system may include artificial intelligence and machine learning capabilities. That is, the system may be capable of allowing the system to learn as more sessions are completed, which may occur through semi-supervised machine learning. This may allow future iterations (e.g., future initiations of the system) to forego or bypass the calibration based on room type or forego or bypass the calibration entirely based on the system building up enough data points to accurately discern between human subjects and other objects (e.g., noise). The system may be provided with machine learning as additional data points become available. Several opportunities may exist for the utilization of machine learning, including, but not limited to reducing the need for an initial calibration prior to each session, enabling a more accurate and appropriate percentage goal for synchronization based on features of the class, the trainer/instructor, and the individual fitness members, as well as the best signals to utilize for accurate return of parameters and variables required for the program to accurately compare the soundwaves via synchronization.

In the case of the initial calibration, as the program gains more data points, a library of echo signatures may be developed to help enable the system to simultaneously filter “noise” during the sessions, as opposed to having to first complete an initial calibration. Machine learning and artificial neural networks may allow the system to more rapidly build a library of echo signatures, thus enabling the program to determine background objects with high efficiency and accuracy. As the binaural spectra aspects of the echoes include the information required to determine the location, size, and shape of objects, the data points from an early set of initial calibrations may be utilized along with machine learning programming such as clustering and pattern recognition. Thus, the machine learning training may include a set of inputs (features from the original sets of initial calibrations) and expected outputs (for example, including, but not limited to accurate labeling of which wavelengths, and respective variables, are indicative of background noise). This may then allow for a model and an algorithm to be built from which future predictions/outputs may occur.

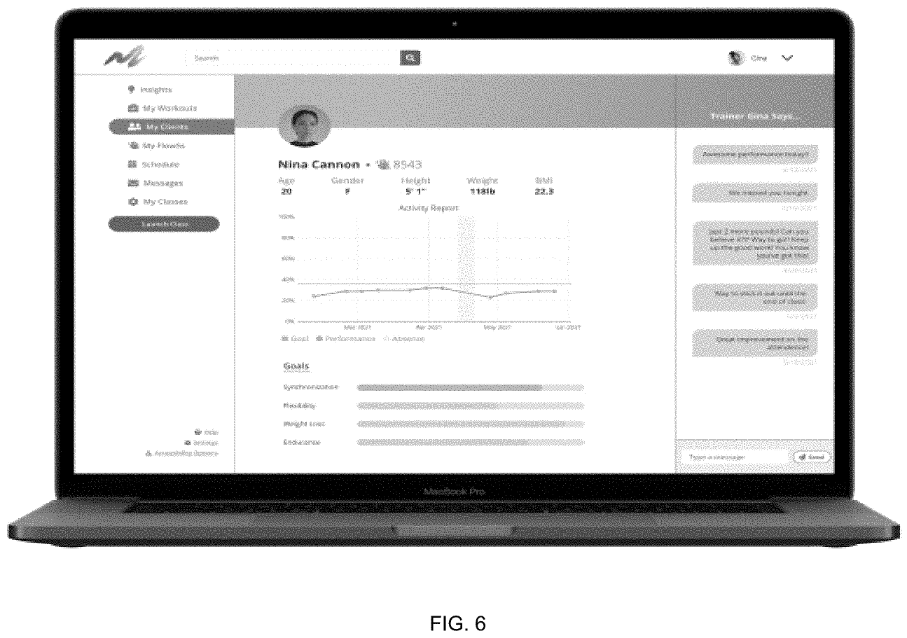

For example, referring to FIGS. 5A-5D, a system process is shown. Generally, as shown in FIG. 5A, the software is initiated, there is an initial calibration step (FIG. 5B), a settings selection step (FIG. 5C), and a session-initiated step (FIG. 5D). Not depicted is a post-session step, which may depict post-session statistics (e.g., FIG. 6). Although depicted in a particular order, the steps may be provided in any order. For example, the settings selection step may occur before the initial calibration. One or more of the steps may be omitted. For example, the settings selection step may be omitted and a default setting applied every session.

Referring to FIG. 5B, the system determines if the software has been initiated and then enters the initial calibration step. Here, either automatically or manually selected by the user, the system enters or skips the initial calibration step. If the initial calibration is denied, then the settings from the most recent calibration are applied to the system. If the initial calibration is initiated, then the system calibrates to the environment, as has been discussed herein, to determine noise created by the user's surroundings (e.g., furniture, appliances, decorations, etc.). Next, the system filters the noise generated by the user's surroundings, either based on a prior calibration or the new calibration.

After calibration, the system proceeds to FIG. 5C for settings selection. The user may opt-in or the system may automatically determine to utilize a) default settings or settings from a prior session or b) the system may provide a number of prompts to create settings for the current session. If default settings are selected, then the default settings are applied and the synchronization settings are initiated. If default settings are not selected, then the user is prompted to select automatic setting selection (e.g., automatic based on class type and/or class level, etc.). If automatic setting selection is selected, then the automatic settings are applied and the synchronization settings are initiated. If automatic setting selection is not selected, then the user is prompted to select the custom setting. The custom setting may allow the user to select a particular percentage of synchronization. In some embodiments, the instructor will perform the settings selection step. In some embodiments, the user will perform the settings selection step. After customized settings are selected, the customized settings are applied and the synchronization settings are initiated.

After calibration and settings are selected, the session may be initiated in step 1, as shown in FIG. 5D. Once the session is initiated, the system may begin receiving soundwaves. The mobile device may emit and/or receive soundwaves. An output device (e.g., a mobile device) may emit the soundwaves and an input device (e.g., a mobile device) may receive the soundwaves. The input device and output device may be the same device. The input device and output device may be different devices. The input and output devices may be dedicated transmitters and receivers, respectively, and may transmit the signal to a mobile device for processing. When the input device receives the soundwave (e.g., acoustic signal, spectral properties) at step 5, the calibrated noise from FIG. 5B may be filtered from the signal. Alternatives and/or additional ways to filter the signal may include low-band, high-band, pass-band filters as well as other computer processing techniques. Filtering signals allows for the user's movements to be tracked without interference from the surrounding environment. The filtered signal is then converted in an analog-to-digital (A/D) converter in step 7 to a digital signal and, in step 8, an input-output controller transfers the signals to a server(s). The digitized signal of the users (e.g., participants and instructor) are compared in step 9 and synchronization percentage is calculated. In step 10, the system determines if the synchronization is within the expected range. If the synchronization is not within the expected range or about the predetermined target level, an alert (step 11) is sent to the instruction, the participant, or both. Steps 2-11 of FIG. 5D are continuously repeated in real-time during the duration of the session. When the session is completed, the soundwaves are no longer emitted and the user is provided with a summary. In the case of the instructor, the summary may include information on individual participants or the class as a whole. In the case of the participant, the summary may include the amount of time the user was within the accepted range of synchronization and/or may include a graph of such information over the time period of the class and/or across each class participated in (FIG. 6). Other analytics may be provided to the user based on the information gathered and processed during the session. FIG. 5E depicts additional process steps that may be employed in the system.

In other words, the session is initiated through the software interface (FIG. 5D, step 1). Then, the transmitter will send an acoustic wave propagation (FIG. 5D, step 2), the specifics of which may be generated from the software interface, thereby emitting the acoustic waves into the air (FIG. 5D, step 3). The output device, or transmitter, will consist of one of the following iterations (see below), some of which exist within the specific computing device (e.g., cell phone, laptop, tablet, iPad, smartphone, desktop computer, etc.) or others which exist outside of the computing device (speakers which are connected either by plug, cable, or Bluetooth).

In some examples, broadband wavelengths may be provided for transmission. Broadbands may be able to more accurately detect object position and orientation, especially when taking temporal properties into account, as in a principal object of the present disclosure. Thus, broadbands may be the most appropriate wavelengths for transmission for real-time synchronization of users' ultrasonic soundwave reflections. Broadbands are also better able to achieve accuracy in terms of the aspects of spectral object components, as broadbands are more effective in cluttered environments, which could include the indoor space of a user's home, hotel room, or the trainer/instructor's fitness studio, etc. Overall, the signal processing bandwidth may be adjusted based on the reflection of the targets, in order to allow for the entirety of the desired signal to be processed. This, in effect, may also benefit from machine learning over the course of the present disclosure.

The software interface will initiate reception of soundwaves (FIG. 5D, step 4). After the soundwave is propagated, the soundwave will pass in part through objects, and be reflected back in part (through echoes) to the receiver (FIG. 5D, step 5). The returning soundwaves will be received by the input device (see below for iterations of receivers). The time from the emission of the soundwave and the reception of the soundwave's echo is scientifically referred to as the Time of Flight (TOF). The TOF measures the proportional distance traveled by the soundwaves.

The soundwaves (analog signals) will pass through a filter (FIG. 5D, step 6), which will distinguish between the unwanted signals (often referred to as noise) and the desired (target) signals. The filter will know which signals are unwanted “noise” due to the signals obtained from the initial calibration. The analog-to-digital (A/D) converter will then translate the filtered acoustic signals that were received through the computing device microphones and convert them to a digitized signal (FIG. 5D, step 7). The input/output (I/O) controller will then transfer the digitized signals to a cloud computing system (FIG. 5D, step 8) for continuous comparison of synchronized, or lack of, between the digitized signals of the instructor versus the digitized signals of the users (FIG. 5D, step 9). Alternatively, the system may send only some user data to the cloud, while other data may be sent from the cloud to the users' device for calculation. If the system detects that any user(s) is not in synchronization with the instructor (based on the digitized signals) (FIG. 5D, step 10) then the software will produce an alert, which will be displayed as a red colored outline surrounding the video of the participant who is not in-synchronization (FIG. 5D, step 11). Alternative methods of alerting the instructor include audio, tactile, or other visual methods such as bringing up a visual image of the user in question on the instructors' device.

In an embodiment, the users (e.g., instructor and participants) may be displayed via a three-dimensional graph, which displays distance (centimeters) versus angular position (degrees) versus time (seconds), used to represent the results of mechanical/acoustic waves interacting with target objects in the environment. In an embodiment, an acoustic fingerprint representing the targets (e.g., instructor and participants) and may be displayed via a two-dimensional graph of spectral target strength, which is frequency (kilohertz) in relation to intensity (relative decibels), versus angular position (degrees). The acoustic fingerprints may be conducted on each primary target (instructor) in real time for the purpose of comparing the synchronization, or lack thereof to the pre-determined degree described in the paragraph above, to each secondary target (fitness members). The comparisons to the aforementioned graphs will be completed on the backend through cloud computing and may not be displayed to the targets.

The system of the present disclosure may rely upon known mathematical models to achieve the desired functions. For example, the fast Fourier transform (FFT) may convert analog and digital signals into spectral, and the time domain into the frequency domain, the output of which may then be utilized in visual representations, such as the graph models mentioned previously. For example, the Known Point Initialization (KPI) algorithm combined with Doppler differential positioning algorithm may utilize the least squares method to determine the user's velocity and location.

Referring to FIG. 6, post-training or after completing a fitness class, both instructors/trainers and participants/trainees may view the results of the previous training sessions/classes and may track progress. For example, a participant may view the predetermined target goal of synchronization and the degree of difference (e.g., measured in percentage) that the user is in synchronization. Other data may be provided as well, including, but not limited to improvement of synchronization over time, percentage of synchronization on average, percentage of synchronization based on class and/or difficulty, etc. An instructor may view these statistics for all participants in a given class, while the participants themselves would be able to view the details of their own statistics unless they choose to share them with their connections within the community as an additional form of accountability and encouragement. Thus, providing another opportunity for follow-up on behalf of the instructor/trainer and another means of progress evaluation and operant conditioning by way of positive reinforcement or positive punishment, depending respectively on the progress made or lack thereof.

The system may include “add-ons” for the users that may increase motivation, such as, for example, through the added level of competition and/or operant conditioning aspect of monetary rewards. For example, a user may set his or her own goals, such as how often one attends fitness classes, percentage of synchronization achieved, etc. If the user reaches the set goals then the user receives an award (monetary or otherwise). The pool of rewards may come from other users who opt-in. For example, each user may contribute $10 per month to hold himself or herself accountable for set goals. If 10 users participate, but only one user meets all of the set goals, then the user who met his or her goals would be awarded $100 for the month. If more than one user meets the goals, the award may be split between the users. Additional iterations may include family members or friends who are interested in a user's health being able to make monthly contributions and the user may receive the payout when the set goals are met. Another iteration may include the user being able to select the award be provided to a charity instead of the community, especially in the case of a family or friend (sponsor) purchasing. For example, if a concerned spouse knew that her husband wanted to buy a new phone, she may set aside $1000 for her husband to meet all goals set within 3 months, and if he reaches his goals, then he would receive the money, enabling him to purchase the phone. Otherwise, if he does not meet those goals, then instead his $1000 would go to a charity partner of her choice. Suggestions for goals and rewards could be curated by a habit questionnaire that may be utilized to determine personality type of the user to optimize the psychological potential of motivation and psychological conditioning. This curated option may be utilized for both individual and/or family members/friends if the user/sponsor is unsure of how best to leverage the technology; custom features may also be available. Overall, the technology may serve as either operant conditioning punishment or positive reinforcement, depending on the users' outcome.

The transmitter and receivers may be placed at any location with respect to the user. Of importance is that the transmitter and receivers are placed at the same relative location for both the instructor and the participants. That is, the center for the instructor and each of the participants may be the same. In an example, this may include the instructor beginning a session by instructing the participants as to where to locate their transmitter/receivers and/or there may be a predetermined location based on the type of class to be performed. A more accurate relative location of the system for the instructor and each respective participant will result in a more accurate indication of the synchronization of movements and thus a more accurate determination of percentage of synchronization.

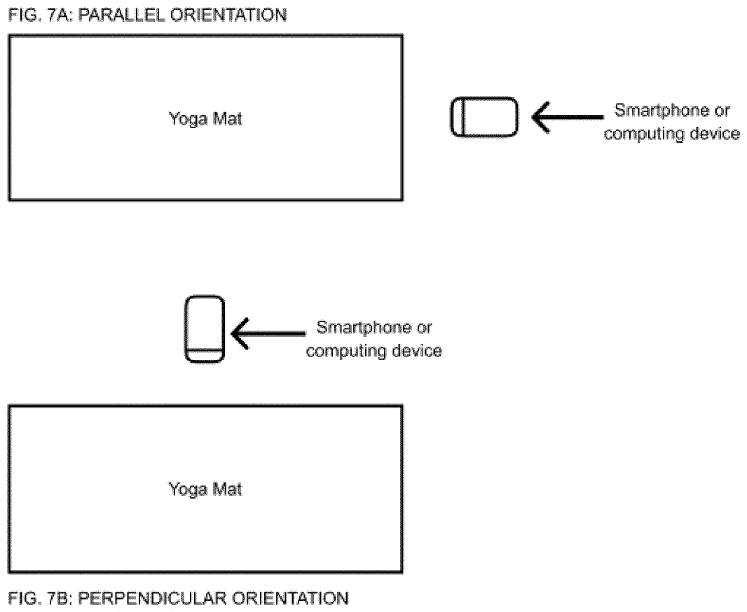

In some embodiments, the receivers may be placed in front of the user. The system is not required to visualize or “see” the users, rather, the system requires the ability to compare their location relative to a designated center. In some particular cases the user may designate a preferred location to place the transmitters/receiver relative to the workout space, for example, in the case of yoga mats the placement of the mat and the individual users may matter, such that the mat should be either parallel (see FIG. 7A) or perpendicular (see FIG. 7B) to the user at the start of the session/lesson and more specifically that the transmitters/receiver be placed for example at the top center of yoga mat (see FIG. 7). Additionally, the software instructions will indicate to users the ideal distance threshold (as with the ideal computer/technical specifications) for users to achieve optimum results.

Accordingly, the system of the present disclosure allows for individuals unable to or unwilling to travel to a physical gym location (e.g., live in a very rural area, be a high-risk individual during pandemic, be quarantined or self-isolated, have limited time, be unable to travel, spend less money, etc.) and utilize home gym equipment or location while still benefiting from community aspects, group support, and personalized coaching. Individual participants may also utilize the present system in a mostly (or always) empty condo/apartment/hotel/resort/office gyms without feeling alone and isolated. The system also allows for augmenting on vacation or traveling for work and needing to utilize unfamiliar gym locations or in situations when a participant is uncomfortable in that setting. Participants may even attend group classes online from a hotel room with no need for a physical gym.

The system of the present disclosure also utilizes the psychology of learning with operant conditioning through the positive reinforcement of congratulatory cues from the instructor/trainer and positive punishment through the instructor verbally indicating in front of the entire group/class when a participant is falling behind or not performing as instructed. Operant conditioning has a much higher correlation to the formation of positive habits than self-reported measures which highly lack validity and reliability. Overall, the system has a lower entry cost (does not require expensive, specialty equipment), is portable (e.g., relying upon a mobile device such as a phone, tablet, or other computing device), is not dependent on ambient light, and resulting in increased privacy, decreased processing power and increased speed as compared to prior art solutions.

The real time monitoring and alerts allow for individuals using the system of the present disclosure to truly retain community, and in turn, increases social connections and decreases loneliness. Prior art solutions (e.g., Peloton®, Mirror®, Tempo®) are virtual communities, which do not allow for members to stay connected to their deeply rooted fitness groups. Research has demonstrated severe health effects due to loneliness, for example, but not limited to, being as detrimental as daily smoking three-quarters of a pack of cigarettes. The present system addresses isolation and loneliness and helps fitness businesses and trainers to stay relevant, profitable, and employed. Positive social and community ties, as well as fitness routines, may help to ameliorate negative health effects and lead to better illness recovery. An adequate amount of exercise has been negatively correlated to loneliness. Participants may continue to attend fitness businesses, but may also continue their fitness journey at home due to constraints, such as, for example, a pandemic, being out of town due to vacation, or business trips. The system may work simultaneously with in-person classes. That is, some participants may be remote, participating virtually, while the trainer or instructor may be in the physical gym location with participants at the gym as well.

In embodiments of the present disclosure one transmitter and two receivers may be present. In embodiments where transducers are employed, two or more transducers may be present. In embodiments, a duplexer may be present. The duplexer may allow the system to switch between transmitting and receiving waves so that the signals are not blocked, interrupted or dampened. In some embodiments, additional hardware (e.g., transmitters/receivers) may be required. In some embodiments, the transmitter and receivers may be present within a computing device. For example, but not limited to, the transmitter and receivers may be present within a combination of 1 and 2, 3, or 4.
1) A computer, laptop, tablet, iPad, or smartphone speaker may act as a transmitter.
2) Most smart phones, including iPhones, have more than one microphone location, two of which may serve as the receivers (e.g., later model iPhones have 3+ microphones).
3) Most iPads/tablets have more than one microphone location, two of which may serve as the receivers (e.g., iPad Air & iPad mini have 2 microphones).
4) Most laptops/MacBooks/desktops/iMacs have more than one microphone location, two of which may serve as the receivers (older laptops may require ancillary speakers).

In embodiments of the present disclosure, the system allows for the measuring of angularity, frequency, velocity, acceleration, or any combination thereof of the user. This information may be provided to the user via the user interface. The user interface may also show whether the participant is meeting or not meeting the parameter (e.g., whether the user has strayed from a predetermined value or range). That is, for example, if the percent of synchronization of the angles, frequency, velocity of acceleration between the participant and the instructor is within a predetermined range or above a predetermined value. The percentage of synchronization may be indicative of the user's ability to perform exercises and movements in proper form. In some embodiments, a beginner's class, such as a beginner's yoga class, may have predetermined ranges of below 30% for unacceptable synchronization and above 50% for excelling synchronization. In some embodiments, an advanced class, such as an advanced boot camp, may have predetermined ranges of below 50% for unacceptable synchronization. In advanced classes, the expectation may be high such that less asynchronization is tolerated, while in beginner classes, the participant is learning and more asynchronization is tolerated.

In some embodiments, the percentage of synchronization showing a failing or falling behind participant in an easy class may be below 10%, below 15%, below 20%, below 25%, below 30%, below 35%, below 40%, or increments thereof. In some embodiments, the percentage of synchronization showing a failing or falling behind participant in a normal or average class may be below 40%, below 45%, below 50%, below 55%, below 60%, below 65%, below 70%, or increments thereof. In some embodiments, the percentage of synchronization showing a failing or falling behind participant in a difficult class may be below 70%, below 75%, below 80%, below 85%, below 90%, or increments thereof. The percentage of synchronization considered unacceptable may change throughout the duration of class schedule. For example, in a first beginner class for yoga, the unacceptable range may be below 20%. In a final beginner yoga class of the sequence, the unacceptable range may be below 50%. This may represent the participants' ability to improve as the session continues. The percentage of synchronization determined to be acceptable may be higher in more difficult class due to the increased ability of users attending more difficult sessions.

Existing research on in-air sonar that focuses on human targets utilizes outside transmitters and receivers, such as satellites and cell phone towers. The existing systems rely on other objects to determine the location of the actual target. That is, for example, the in-air sonar may rely on the position of a human or other object to determine the location of another object. The present disclosure relies on the same human to determine the location of the human. That is, the in-air sonar determines the relative location of the target human, not a third party. The present disclosure relates to a system using in-air sonar with a location of reference as the indoor environment that may include an indoor transmitter and receiver and human targets.

Although described for implementation in fitness classes, the present system may be provided in other industries, such as, for example, but not limited to, dance schools, karate classes, sports, academic and educational class, aiding persons with disabilities, other instructional classes, home health, search and rescue, and other situations where monitoring of a person's movements and relative locations of body parts may be desired. The present system may be provided in situations where monitoring of objects and synchronization of data sets in real time is applicable, such as with agriculture and environmental services. For example, the present system may be provided in recycling and garbage retrieval in neighborhoods. That is, an algorithm to pick up the recycling bins, garbage pails when the program is initiated and the objects match the size and scale (are in synchronization with desired outcome).

For example, the target users may be, but are not limited to: persons with disabilities, fitness club members who travel for business, who relocated or recently moved, those with immunodeficiencies, or during flu season, when one is still contagious but feeling well enough to exercise, but not wanting to potentially spread a virus, agoraphobia or other anxiety disorders (which may be likely to stay increased for period post-pandemic) and may help to be a first step towards integrating back to community, persons involved in exposure therapy, visually impaired individuals, and hearing impaired individuals. For example, visually impaired individuals may slowly learn yoga from the feedback of the system of the present disclosure which initially may be individualized (if user required more continuous feedback, which may be a mix of computer program generated feedback and instructor/trainer feedback) before feeling comfortable to attend in-person yoga classes. For example, hearing impaired may have difficulty in knowing when to move out of yoga pose, for example when completing any poses that are not forward facing poses, therefore, an additional embodiment may allow a user to elect to have a vibration sent to one's phone or other mobile device to alert said user that the pose has changed so that said user may be alerted to visually look at the screen to determine what the instructor is doing at that given point. For some individuals with an emotional, psychological, or physical disability, the accommodations contemplated by the system of the present disclosure allows a larger portion of the population to benefit from socialization, community, and the health benefits afforded by these technologies as without this technology, these individuals may not otherwise join fitness groups.

In examples where the ultrasonic wave synchronization system employs Doppler, additional processing may be required. For example, all wavelengths aside from those that correspond to human tissue may need to be removed—thus, ignoring the “noise”. In some embodiments, a filter may be applied along with continuous spectral Doppler waves. The filter may remove the mechanical/acoustic wave reflections that are at a wavelength representative of a nonhuman tissue versus human tissue, thus reducing the “noise” and increasing the accuracy between targets/users (instructors and participants) comparisons. The filters may allow certain frequency wavelengths reflections to be analyzed, while blocking the frequencies deemed to be “noise.” In some embodiments, a user may wear clothing embedded with Reconfigurable Intelligent Surfaces (RISs) to improve Doppler signals. Furthermore, Doppler signals require piezoelectric crystals and add-on hardware may be required. Within Doppler signals themselves, continuous spectral Doppler may be more promising as Doppler may continually send and receive soundwaves, whereas pulsed-form spectral Doppler may require discrete transmitters and receivers in order to propagate the soundwaves.

While various embodiments of the present invention have been described above, it should be understood that they have been presented by way of example only, and not limitation. Features, in whole or in part, in one embodiment may be utilized in other embodiments. Thus, the breadth and scope of the present invention should not be limited by any of the above-described embodiments but should instead be defined only in accordance with the following claims and their equivalents.

1. An ultrasonic wave synchronization system, comprising:
an acoustic system configured to measure a first parameter of a first user and a second parameter of a second user,
wherein the first parameter is compared to the second parameter to determine a percentage of synchronization between the first user and the second user.

2. (canceled)

3. The ultrasonic wave synchronization system of claim 1, wherein the first parameter and the second parameter result from ultrasonic sound waves reflecting off the first user and the second user, respectively.

4. The ultrasonic wave synchronization system of claim 1, wherein the first parameter is compared to the second parameter to determine closeness of the parameters and wherein the percentage of synchronization is representative of the closeness of the parameters.

5. (canceled)

6. The ultrasonic wave synchronization system of claim 1, wherein the first parameter and the second parameter are one or more of angularity, frequency, velocity, and acceleration and wherein the acoustic system measures the first and second parameter by transmitting and receiving ultrasonic soundwaves.

7. The ultrasonic wave synchronization system of claim 1, wherein the primary user is an instructor and the second user is one or more participants, and wherein the percentage of synchronization is representative of an amount of synchronization of body movements of each of the one or more participants with respect to body movements of the instructor during a fitness class.

8. The ultrasonic wave synchronization system of claim 7, wherein the one or more participants and the instructor are alerted to at least one of the one or more participants that are below a predetermined target of synchronization with the instructor.

9.-12. (canceled)

13. The ultrasonic wave synchronization system of claim 1, further comprising a transmitter, wherein the transmitter and two receivers for the first user are located at the same respective location to the first user as the transmitter and two receivers of the second user.

14. (canceled)

15. The ultrasonic wave synchronization system of claim 1, wherein the first parameter is compared to the second parameter in real-time such that the comparing occurs simultaneously and continuously while the first user and the second user are engaged in an activity.

16.-22. (canceled)

23. A computer-implemented method of determining synchronization between users, the method comprising:
(a) transmitting and receiving a first signal;
(b) determining a first parameter of a first user based on the received first signal;
(c) transmitting and receiving a second signal;
(d) determining a second parameter of a second user based on the received second signal;
(e) comparing, with a processor, the first parameter to the second parameter to determine whether the first parameter and the second parameter are within a predetermined range of one another; and
(f) determining, with the processor and based on the comparison, a percentage of synchronization of the first user and the second user.

24. The method of claim 23, further comprising alerting one of the first user and the second user when the percentage of synchronization is outside of a predetermined range.

25. The method of claim 24, wherein the alerting is a visual alert on a display unit or an audio alert or a tactile alert or some combination thereof.

26. The method of claim 23, further comprising continuously performing steps (a)-(f).

27. The method of claim 23, wherein steps (a)-(f) are performed in real-time.

28. The method of claim 23, wherein steps (a)-(f) are repeated during a timed session and the percentage of synchronization is determined continuously through the timed session.

29. The method of claim 28, further comprising graphing the percentage of synchronization as compared to a predetermined target is presented during and/or after the timed session and providing a resulting graph to at least one of the first user and the second user.

30. The method of claim 28, wherein the primary user is an instructor and the secondary user is one or more participants, and wherein the percentage of synchronization is representative of an amount of synchronization of body movements of each of the one or more participants with respect to body movements of the instructor during the fitness class.

31. The method of claim 30, further comprising tracking an ability of the one or more participants to achieve synchronization within a predetermined range over the course of one or more fitness classes.

32.-36. (canceled)

37. The method of claim 23, wherein the transmitting and receiving of the first signal is provided by a first computing device and the transmitting and receiving of the second acoustic signal is provided by a second computing device, and wherein the first computing device is a personal computer, a mobile phone, or a tablet and wherein the second computing device is a personal computer, a mobile phone, or a tablet.

38. A method for determining synchronization between multiple users, the method comprising:
providing a system to monitor the multiple users; and
comparing, with a processor, the parameters of the system to determine a percentage of synchronization between at least two of the multiple users.
